(19) United States
(12) Patent Application Publication
(10) Pub. No.: US 2025/0147044 A1
(43) Pub. Date: May 8, 2025

(54) ARTIFICIAL INTELLIGENCE (AI) -BASED METHOD FOR IMMUNOGENICITY ASSESSMENT

(71) Applicant: Subhadra Dravida, Hyderabad (IN)

(72) Inventor: Subhadra Dravida, Hyderabad (IN)

(73) Assignee: TRANSCELL ONCOLOGICS PRIVATE LIMITED, Hyderabad (IN)

(21) Appl. No.: 19/017,620

(22) Filed: Jan. 11, 2025

Related U.S. Application Data
(63) Continuation-in-part of application No. 18/235,895, filed on Aug. 21, 2023.
(63) Continuation-in-part of application No. 17/722,528, filed on Apr. 18, 2022.

(30) Foreign Application Priority Data
Feb. 15, 2022 (IN) 202241008032

(51) Int. Cl. G01N 33/68; C12N 5/0781; C12N 5/0783; G01N 33/50

(52) U.S. Cl. CPC G01N 33/6863; C12N 5/0635; C12N 5/0636; G01N 33/505; G01N 33/5052; G01N 2500/10

(57) ABSTRACT

The present invention discloses an artificial intelligence (AI)-based method for immunogenicity assessment. The method involves providing an in-vitro human hematopoietic microphysiology system (hMPS) to simulate a microenvironment that mimics the human hematopoietic system for evaluating immune responses to specific drug. The hMPS is analyzed using various laboratory instruments. The laboratory instruments provide an input data having phenotypic and biochemical responses. The inputs are evaluated by an AI system comprising AI powered in silico tools. The AI system is trained with a machine learning (ML) predictive model using a plurality of training datasets. Further, the platform enables the AI system to perform both qualitative and quantitative analyses of the drug's immune responses using benchmark patterns. The AI analyzes the qualitative and the quantitative data to produce a detailed report on immunogenicity.

### CROSS-REFERENCE TO RELATED APPLICATIONS

The present application is a continuation-in-part application of patent application Ser. No. 18/235,895 titled “DIGITAL ANIMAL FREE TESTING (DAFT) SYSTEM” filed on Aug. 21, 2023 in the United States Patent and Trademark Office, which is a continuation-in-part application of patent application Ser. No. 17/722,528 titled “A NON-ANIMAL HUMAN RELEVANT WORKSTATION SYSTEM AND METHOD FOR TESTING NEUROVIRULENCE AND NEUROTOXICITY IN VACCINES”, filed in the United States Patent and Trademark Office on Apr. 18, 2022, which further claims priority to Indian patent application Ser. No. 20/224,1008032 filed on Feb. 15, 2022, the entire contents of which are hereby incorporated by reference for all purposes.

### TECHNICAL FIELD

The present invention generally related to an immunogenicity assessment. More particularly, the present invention is related to an artificial intelligence (AI)-based method for immunogenicity assessment.

### BACKGROUND

Immunogenicity is an ability of a substance including a drug or vaccine to provoke an immune response when introduced into an animal model. Immunogenicity assessments have traditionally relied on the animal model to predict human immune responses, as animal models provide a controlled environment for studying the interaction between the immune system and the test substance. The immunogenicity in the models enable researchers to examine immune activation, efficacy of the therapeutic agent, adverse reactions, tolerability, and safety of the drug.

However, the immunogenicity analysis using animal models could be time-consuming, incur high costs, ethically challenging, and often involves complex procedures. The traditional animal models often fail to accurately predict human immune responses that leads to ethical and financial concerns. The use of animals in research that does not lead to reliable human outcomes can be seen as a misuse of resources and may involve unnecessary suffering for the animals, particularly when alternative methods could provide more accurate results. Further, significantly differences in immune system structures between species could alter their interactions with therapeutic agents. The differences between animal and human immune systems could lead to non-predictive or unreliable results.

Therefore, there is a need for an immunogenicity assessment method that needs to be convenient, time-efficient, and capable of addressing ethical challenges. The method needs to eliminate the need for complex procedures and needs to provide accurate and clinically relevant insights into immune responses. Additionally, the method needs to be able to provide a safer and more effective drug development and reduce the reliance on animal testing.

### SUMMARY

The present invention discloses an artificial intelligence (AI)-based method for immunogenicity assessment. The method is utilized to measure efficacy of specific drug or test substance and ensure the specific drug induces a robust and protective immune response. At one step, an in-vitro human hematopoietic microphysiology system (hMPS) is provided. The hMPS is configured to simulate a microenvironment that mimics a human hematopoietic system. The in-vitro hMPS incorporates bioassay processes to evaluate the function of the miniature hematopoietic system using the specific drug. At another step, the hMPS is analyzed using various laboratory instruments. The laboratory instruments capture a phenotype and a biochemical response from the treated in-vitro hMPS. At yet another step, one or more input data from laboratory instruments operating in combination with the in-vitro hMPS are provided to an AI system. The input data is generated in various format including micrographs, flow cytometry graphs, image datasets, and numerical data.

At yet another step, one or more input data including the phenotypic and the biochemical response are evaluated utilizing the AI system or platform comprising AI powered in silico tools The AI system is trained with one or more immunogenicity prediction models having a plurality of training datasets. The training dataset is generated from a collection of relevant controls, references, and standards panels treated within in-vitro system during the bioassay process. The control is an untreated sample without drug treatment. At yet another step, at least one set of benchmark patterns are generated for each training dataset using signals from control, reference, and standard panels of the specific drug using the AI system. At yet another step, the AI system performs qualitative and a quantitative analysis of the specific drug using the benchmark patterns, and analyze the qualitative and the quantitative data to produce a detailed report on immunogenicity.

The method is an essential factor in the development of one or more biopharmaceuticals, particularly biotherapeutics and vaccines. In one embodiment, the specific drug comprising an innovator drug and a test biosimilar, along with a TGF-β inhibitor, are tested utilizing the in-vitro hMPS to assess the immune response to the drug in two distinct sets. The innovator drug is taken as a reference standard for comparison with the biosimilar drug. The effect of the innovator drug and the test biosimilar shows increased expression of a biomarker including CD80 and CD86 indicating the activation of the antigen-presenting cells (APCs), and increased cytokine levels in response to both the innovator drug and the test biosimilar by a third day. The effect of the innovator drug and the test biosimilar shows increased plasma cell numbers and IgG in response to both the innovator drug and the test biosimilar by a fifth day. The dataset from the innovator drug and test biosimilar exhibit less than 15% variation, thereby meeting established clinical quality standards.

In another embodiment, the specific drug comprising a standard vaccine and a test vaccine, are tested using in-vitro hMPS to assess the immune response to a bacterial pathogen in two distinct sets. The effect of the standard vaccine and the test vaccine shows increase of an anti-inflammatory cytokine including IL-10 and IL-4 and decrease in the expression of pro-inflammatory cytokines including IFN-gamma, TNF-alpha, and IL-6, thereby preventing trigger of excessive inflammation in response to both the reference standard vaccine and the test vaccine. Further, the effect of the reference standard vaccine and the test vaccine shows increased plasma cell numbers in response to both the reference standard vaccine and the test vaccine by the fifth day. The effect of the reference standard vaccine and the test vaccine shows increased Immunoglobulin G (IgG) in response to both the reference standard vaccine and the test vaccine. The method further comprises step of co-culturing the enriched antigen-presenting cells (APCs) with T cells and B cells in a specific ratio designed to mimic physiological conditions. The efficacy of co-culture supernatants was assessed via a bactericidal assay and AI system for both the reference and test vaccines. Both the reference and test vaccines achieve a titre of 16 indicating effective antibacterial potential.

### BRIEF DESCRIPTION OF THE DRAWINGS

FIG. 1 illustrates a flowchart of an artificial intelligence (AI)-based method for immunogenicity assessment, according to an embodiment of the present invention.

FIG. 2 is a flowchart showing an immune response analysis used in the assessment of immunogenicity, according to an embodiment of the present invention.

FIG. 3 illustrates a block diagram for assessing immunogenicity in vaccines and biotherapeutics, according to an embodiment of the present invention.

### DETAILED DESCRIPTION OF EXAMPLE EMBODIMENTS

The present invention discloses an artificial intelligence (AI)-based method for immunogenicity assessment. The method utilizes a digital animal free testing (DAFT) strategy for the immunogenicity assessment. The DAFT strategy is an innovative new approach methodology (NAM) that aligns with the definitions established by a food and drug administration (FDA) and the Center for Drug Evaluation and Research (CDER) under the Modernization Act 2.0. The cutting-edge technology addresses critical challenges in biopharmaceutical development and provides a robust solution for non-clinical immunogenicity assessments during both developmental and batch-testing stages.

The DAFT strategy serves as an ethical alternative for evaluating vaccine efficacy and assessing safety risks associated with biosimilar candidates. Additionally, the DAFT strategy reduces variability and enhances the statistical significance of findings related to human health outcomes. Further, the DAFT strategy is used in evaluating therapeutic immunogenicity, testing candidates for new vaccines, and investigating potential drug toxicity profiles. The DAFT strategy adheres to the principles of 3Rs that includes Replacement, Reduction, and Refinement in animal testing by producing human-relevant responses, which promotes ethical research practices. The DAFT approach enables more accurate and clinically relevant insights into immune responses by employing an advanced in-vitro human hematopoietic microphysiology system (hMPS) and an artificial intelligence (AI).

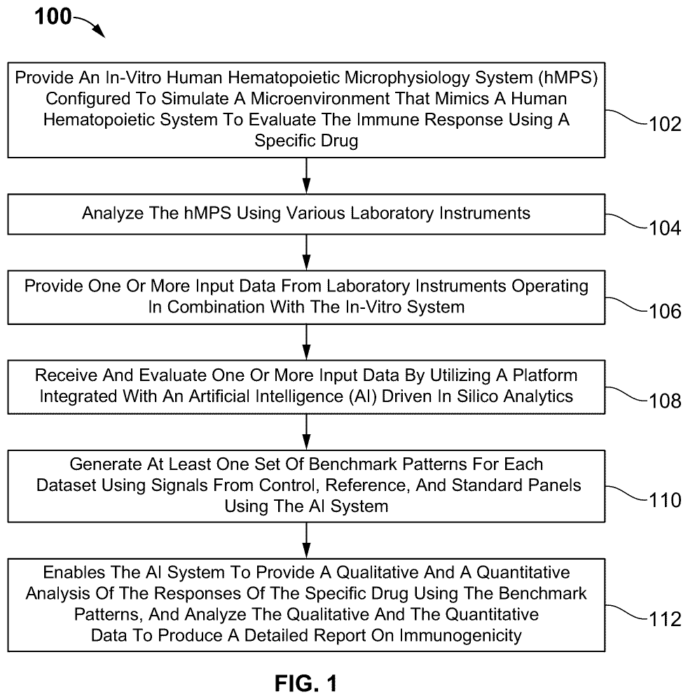

The AI approach addresses limitations of traditional methods including ethical dilemmas, high costs, and inconsistent results due to the inability of an animal models to replicate a human immune system. FIG. 1 illustrates a flowchart 100 of an artificial intelligence (AI)-based method for immunogenicity assessment, according to an embodiment of the present invention. The method is utilized to measure efficacy of specific drug or test substance and ensure the specific drug induces a robust and protective immune response. The method involves an in-vitro human hematopoietic microphysiology system (hMPS) to simulate a microenvironment that mimics a human hematopoietic system. The hMPS is treated by incorporating bioassay processes using a test substance. Further, input data including a phenotype and a biochemical response from the treated in-vitro hMPS is collected using one or more laboratory instruments. The input data including the phenotypic and the biochemical response is analyzed by utilizing an AI system comprising AI powered in silico tools. Thereafter, a comprehensive immunogenicity report generated using the AI system.

At step 102, an in-vitro human hematopoietic microphysiology system (hMPS) is configured to simulate a microenvironment that mimics a human hematopoietic system. The in-vitro hMPS incorporates bioassay processes to evaluate the function of the miniature hematopoietic system using the specific drug.

At step 104, one or more laboratory instruments are configured to analyze the hMPS. The laboratory instruments are configured to capture a phenotype and a biochemical response from the treated in-vitro hMPS. The laboratory instruments include, but not limited to, microscopes, flow cytometers, and ELISA systems.

At step 106, the laboratory instruments operating in combination with the in-vitro hMPS provides one or more input data to an AI system. The input data is generated in various formats includes, but not limited to, micrographs, flow cytometry graphs, image datasets, and numerical data. The input data enable a thorough evaluation of the biological systems under investigation.

At step 108, the input data including the phenotypic and the biochemical response are evaluated utilizing the AI system or platform comprising AI powered in silico tools. The AI system is a framework that employs artificial intelligence (AI)-driven in silico analytics to generate comprehensive insights from the in vitro bioassays. The AI system is trained with one or more machine learning (ML) predictive models having a plurality of training datasets. The training datasets are obtained from a collection of relevant controls, references, and standards of the specific drug treated within in-vitro hMPS during the bioassay process. Further, the method systematically combines phenotypic and biochemical evaluations within the machine learning (ML) predictive model that assists the AI system in performing an extensive analysis of immunological responses. In one embodiment, the machine learning (ML) predictive model is an immunogenicity prediction model.

At step 110, at least one set of benchmark patterns are generated for each training dataset using signals from control, reference, and standard panels of the specific drug and AI system. At step 112, the AI system uses the benchmark patterns to perform a qualitative and a quantitative analysis of the specific drug. Further, the AI system analyze the qualitative and the quantitative data to produce a detailed report on immunogenicity. The report is capable of automatically generating detailed and user-friendly reports. The report eliminates the need for cumbersome manual analysis and enhances the efficiency, accuracy, and reliability of the reporting process. The robotic automation of immunogenicity assessment allows a researcher to focus more on critical decision-making regarding the optimization and strengthening of a therapeutic pipeline.

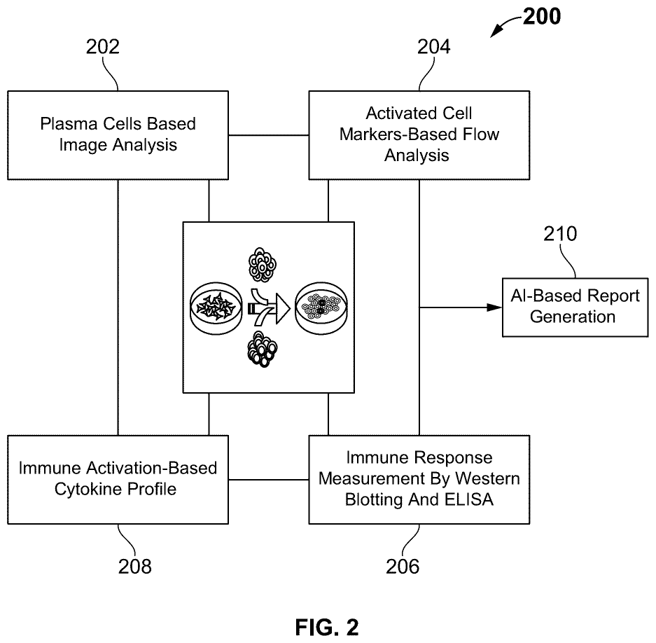

FIG. 2 is a flowchart 200 showing an immune response analysis used in the assessment of immunogenicity, according to an embodiment of the present invention. At step 202, the method for immunogenicity assessment involves a plasma cell-based image analysis. The plasma cell-based image analysis employs advanced imaging techniques to evaluate the plasma cell morphology, density, and phenotypic characteristics under experimental conditions.

At step 204, the method for immunogenicity assessment involves an activated cell markers-based flow analysis. The activated cell markers-based flow analysis utilizes flow cytometry to identify and quantify activated immune cells by analyzing specific surface and intracellular markers.

At step 206, the method for immunogenicity assessment involves an immune response measurement. The immune response measurement involves biochemical assays, for example, a western blotting and an enzyme-linked immunosorbent assay (ELISA). The western blotting is utilized to for detecting proteins. The ELISA is utilized to quantifying immune mediators like cytokines or antibodies. The biochemical assay helps to measure the immune responses.

At step 208, an immune activation-based cytokine profile uses cytokine profiling techniques, for example, ELISA or multiplex assays. The cytokine profiling techniques is utilized to measure cytokine levels and evaluate the immune activation state.

At step 210, the method for immunogenicity assessment involves generating an AI based report. The AI based report is produced by feeding the data obtained from step 202, step 204, step 206, and step 208 into the machine learning (ML) prediction model. The machine learning models provides the comprehensive analysis and generates a detailed immunogenicity report. Further, the circular flow illustrates the interconnection between the workflows in the flowchart 200. The flowchart 200 highlights the collective role in providing a comprehensive understanding of immune system dynamics.

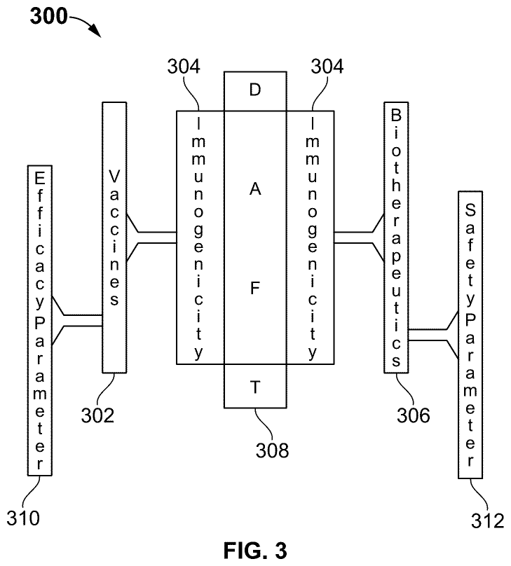

FIG. 3 illustrates a block diagram 300 for assessing immunogenicity in vaccines and biotherapeutics, according to an embodiment of the present invention. The immunogenicity testing is an essential factor in the development of one or more biopharmaceuticals, particularly biotherapeutics and vaccines, at block (302, 304, 306). The flowchart is an outline for evaluating immunogenicity as a critical quality attribute (CQA) of the vaccines and the biotherapeutics. Immunogenicity is the ability to elicit an immune response and is assessed for efficacy in vaccines and safety in biotherapeutics. For vaccines, the immunogenicity process is assessed as a measure of efficacy and ensures the vaccine induces a robust and protective immune response. For biotherapeutics, the immunogenicity process monitors unintended immune reactions, for example, the formation of anti-drug antibodies (ADA).

At block 308, the immunogenicity assessment process is supported by Digital Animal Free Testing (DAFT) strategies or digital animal replacement technology. The DAFT strategy is used to analyze immunological data to validate biopharmaceuticals performance. The DAFT strategy serves as a robust and ethical solution for assessment that replaces traditional animal models with sophisticated in-vitro hMPS combined with AI. The DAFT strategy excels in providing clinical-relevant assessments. Further, the DAFT strategy ensuring that the assessments applied to human health is align with global regulatory efforts to phase out animal testing. Further, the data analysis and reporting tools play a role in analyzing and reporting immunogenicity-related safety data. The ML embedded in silico platform are used to process and interpret immunogenicity data. At blocks (310, 312), the method highlights the dual role of immunogenicity testing in supporting both product development and regulatory compliance, bridging efficacy and safety assessments through advanced in vitro and in silico tools.

The integration of AI system enhances the precision and reliability of immunogenicity evaluations, leading to accurate data analyses and informed predictions relating to safety and efficacy. The NAM facilitates tailored testing processes, allowing for specific assessments that cater to the unique needs of biosimilars and vaccines. The NAM under discussion leverages advanced AI combined with predictive analytics to analyze large datasets and generate detailed the immunogenicity profiles. The AI system integration significantly enhances scalability and accuracy regarding immune response predictions, including the formation of ADAs, when compared to traditional manual analysis techniques.

In one embodiment, the method is utilized to provide the immunogenicity assessment for an innovator drug and a test biosimilar. The immunogenicity assessment is assessed carefully to prevent adverse immune reactions by ensuring patient safety as a paramount. Unintended immune responses lead to significant issues, for example, autoimmune diseases. In autoimmune diseases, the body's immune system mistakenly attacks the own tissues of the body. Thus, a comprehensive understanding of immunogenicity is crucial for safely using biosimilars in clinical settings. The innovator drug serves as a critical reference standard for the test biosimilars. The innovator drug provides a reliable baseline against which the test biosimilar products is to be compared. The comparison is essential for evaluating the safety of biosimilars in relation to the reference standards.

In the biosimilar, the human hematopoietic in hMPS is treated with the innovator drug and the test biosimilar having a TGF-β inhibitor. The treatment activates an antigen-presenting cells (APCs) as indicated by increased expression of biomarkers including CD80 and CD86. By third day, the measurement of cytokine levels including TNF-α and IFN-γ in the treated cultures showed a significant increase in the immune response by the third day. The treated culture comprises of APCs, T cells, and B cells. By the fifth day, the plasma cell numbers increased by 10% with the reference drug and by 20% with the test biosimilar which is determined from micrographs and flow scatter plots reading CD138. Additionally, the levels of the immunoglobulin G (IgG) quantified from the co-culture supernatants were 22% higher for the reference drug and 25% higher for the test biosimilar compared to the control group. Analysis of the datasets from the reference drug and the test biosimilar showed a variation of less than 15%, which is within the established limits and meets the necessary quality standards for clinical use.

In another embodiment, the method is utilized to provide the immunogenicity assessment for a reference standard vaccine and a test vaccine. The vaccines focus on efficacy, with the goal of inducing a strong and protective immune response against specific pathogens. Effective immunogenicity in vaccines is designed to stimulate the immune system. Further, the effective immunogenicity in vaccines enables the immune system to recognize and combat pathogens more effectively if encountered in the future. Therefore, a robust immunogenic profile is needed to ensure the vaccine elicits a strong response and provides long-lasting immunity.

In the vaccine, the human hematopoietic in hMPS is treated with the reference standard vaccine and the test vaccine to assess the immune response to a bacterial pathogen. Further, the enriched APCs in the experimental setup were carefully co-cultured with T cells and B cells in a specific ratio designed to mimic physiological condition. The effect of the treatment showed an increase in anti-inflammatory cytokines, specifically IL-10 and IL-4, along with a decrease in the expression of pro-inflammatory cytokines including IFN-gamma, TNF-alpha, and IL-6. The response pattern was similar for both the reference standard and the test vaccine. Which implies that the test vaccine possesses the ability to stimulate a robust immune response without triggering excessive inflammation that could potentially lead to harmful outcomes for the host.

On the fifth day of the bioassay, a significant increase in the population of the plasma cells is observed. Specifically, there is a 43% increase in plasma cells for the reference standard, while the test vaccine candidate prompted an even greater increase of 50%. The results were quantified through detailed in silico analyses using the micrographs and the flow cytometry scatter plots, allowing for precise measurements of the cellular responses.

Further, the concentrations of immunoglobulin G (IgG) present in the co-culture supernatants were measured to assess the humoral immune response. The data revealed an increase of 52% in IgG levels for the reference standard and a 55% increase for the test vaccine when compared to the control group within the assay system.

Additionally, the efficacy of the co-culture supernatants in eliminating the targeted bacterial pathogen is evaluated by measuring cellular responses using a bactericidal assay and AI system. Remarkably, both the reference standard vaccine and the test vaccine demonstrated a bactericidal titre of 16. The titre level is widely recognized as protective, indicating the potential of both the reference standard vaccine and the test vaccine to effectively combat bacterial infection.

Advantageously, the method is designed with the end-user in mind and is seamlessly integrated into the existing workflows of research and development teams. The integration streamlines the entire process that simplifies the adoption and implementation of the technology, which enables the organizations to share insights at every stage of the product lifecycle. Overall, the non-animal NAM represents a significant leap forward in pursuing safer and more effective medical treatments by prioritizing ethical standards in research.

The method that involved the digital animal free testing (DAFT) strategy focuses on data acquisition from human-relevant in-vitro models treated with biomolecules, thereby ensuring the compliance with ethical standards in research. The DAFT strategy approach facilitates greater efficiency in immunogenicity assessment by integrating in silico tools including AI and Machine Learning (ML). The in-silico integration enhances overall efficiency compared to traditional standalone cell assay techniques. The DAFT strategy integrated with in silico tools reducing the costs associated with animal trials and shortening the timelines for obtaining results enables researchers to speed up the projects without compromising quality or reliability. The efficiency in immunogenicity assessment ultimately led to faster innovation and a quicker, affordable path to market for new therapies.

The DAFT strategy represents a groundbreaking advancement in immunogenicity testing, significantly improving upon the limitations commonly associated with the animal testing models. The AI technology to eliminate the ethical concerns associated with using live animals in research and aligns with contemporary values surrounding animal welfare. Further, the AI technology has an ability to deliver precise insights directly relevant to human application. Further, the AI system ensures that the data generated during the testing process is accurate and enhances the predictability of safety and efficacy for new drug. As a result, researchers and biopharmaceutical companies could make more informed decisions throughout the drug development process and implement the same strategy for product batch release criteria.

The method offers a comprehensive solution amalgamating multiple technologies. For instance, flow cytometry is utilized for immune marker profiling, while ELISA is utilized to evaluate cytokine levels in deriving insights into immunogenicity. The multifaceted approach enhances the versatility of the immunogenicity assessment and increases its translational relevance to real-world clinical applications. The method involved in evaluating therapeutic immunogenicity, testing for new vaccines, and investigating potential drug toxicity profiles. Another notable feature is the integration of real-world experimental data with predictive modelling. The predictive model enables dynamic simulations that effectively bridge empirical observations with advanced computational methodologies.

The method improves high-throughput immunogenicity assessment by enabling the seamless integration of multiple assays into a coherent and efficient workflow within a single module. The integrated of multiple assays not only streamlines the testing process but also improves the reliability and accuracy of immunogenicity evaluations. As a result, the method that integrates multiple assays contributes to advancing the understanding of immune responses in relevant biological contexts.

We claim:

1. An artificial intelligence (AI)-based method for immunogenicity assessment, comprising the steps of:
providing an in-vitro human hematopoietic microphysiology system (hMPS) configured to simulate a microenvironment, wherein the microenvironment mimics a human hematopoietic system;
treating the hMPS by incorporating bioassay processes using a test substance;
collecting input data including a phenotype and a biochemical response from the treated in-vitro hMPS using one or more laboratory instruments;
analyzing the input data including the phenotypic and the biochemical response by utilizing an AI system comprising AI powered in silico tools, and
generating a comprehensive immunogenicity report using the AI system.

2. The AI-based method of claim 1, wherein the AI system is trained with one or more machine learning (ML) predictive models having plurality of training datasets; the AI system is configured to:
generating at least one set of benchmark patterns for each training dataset using signals from control, reference, and standard panels of the test substance, and
enable the AI system to perform a qualitative and a quantitative analysis of the specific substance using the benchmark patterns, and analyze the qualitative and the quantitative data to produce the detailed report on immunogenicity,
wherein the training dataset is generated from a collection of relevant controls, references, and standards panels treated within in-vitro system during the bioassay process.

3. The method of claim 1, wherein the training dataset is generated from a collection of relevant controls, references, and standards panels treated within in-vitro system during the bioassay process.

4. The method of claim 1, wherein the test substance comprising an innovator drug and a test biosimilar, along with a TGF-β inhibitor, are tested using the in vitro hMPS to assess immune responses in two distinct sets, wherein the innovator drug serves as a reference standard for comparison with the biosimilar.

5. The method of claim 4, wherein an effect of the innovator drug and the test biosimilar shows increased expression of a biomarker including CD80 and CD86 indicating an activation of the antigen-presenting cells (APCs), and increased cytokine levels in response to both the innovator drug and the test biosimilar.

6. The method of claim 4, wherein an effect of the innovator drug and the test biosimilar shows increased plasma cell numbers in response to both the innovator drug and the test biosimilar, wherein the effect of the innovator drug and the test biosimilar shows increase of an Immunoglobulin G (IgG) in response to both the innovator drug and the test biosimilar, and wherein a dataset from the innovator drug and test biosimilar exhibit less than 15% variation, thereby meeting established clinical quality standards.

7. The method of claim 1, wherein the test substance is a standard vaccine and a test vaccine, both are tested using the human hematopoietic in-vitro system to assess the immune response to a bacterial pathogen in two distinct sets, wherein the standard vaccine is taken as a reference standard for comparison with the test vaccine.

8. The method of claim 7, wherein an effect of the standard vaccine and the test vaccine shows increase of an anti-inflammatory cytokine including IL-10 and IL-4 and decrease in the expression of pro-inflammatory cytokines including IFN-gamma, TNF-alpha, and IL-6, thereby by preventing triggering of excessive inflammation in response to both the reference standard vaccine and the test vaccine.

9. The method of claim 7, wherein an effect of the standard vaccine and the test vaccine shows increased plasma cell numbers in response to both the reference standard vaccine and the test vaccine, wherein an effect of the standard vaccine and the test vaccine shows increased Immunoglobulin G (IgG) in response to both the reference standard vaccine and the test vaccine.

10. The method of claim 7, further comprises step of: co-culturing the enriched antigen-presenting cells (APCs) with T cells and B cells in a specific ratio designed to mimic physiological conditions, wherein the efficacy of co-culture supernatants was assessed via a bactericidal assay and AI system for both the reference and test vaccines, wherein both the reference and test vaccines achieve a titre of 16, thereby indicating effective antibacterial potential.
